(19) United States
(12) Patent Application Publication
(10) Pub. No.: US 2024/0221861 A1
(43) Pub. Date: Jul. 4, 2024

(54) Apparatus and method for blocking and delivering electromagnetic energy

(71) Applicant: Paul Durieux, Brownsburg-Chatam, QC (CA)

(72) Inventor: Paul Durieux, Brownsburg-Chatam, QC (CA)

(21) Appl. No.: 18/573,139

(22) PCT Filed: Jun. 21, 2022
(86) PCT No.: PCT/CA2022/050995

§ 371 Date: Dec. 21, 2023

Related U.S. Application Data
(60) Provisional application No. 63/202,701, filed on Jun. 21, 2021.

(51) Int. Cl. G12B 17/02

(52) U.S. Cl. CPC G12B 17/02

(57) ABSTRACT

An apparatus in the form of a multi-layered mat and of a multi-layered tile for protection against geopathic radiations in the electromagnetic frequencies (EMF) is provided, the apparatus comprising a combination of a plurality of layers: a first layer against high-frequency radiations, a second layer for blocking medium frequency radiations, a third layer for blocking medium to high frequency radiations, a fourth layer delivering pulsed electromagnetic frequencies (PEMF) and a fifth layer for capturing low level EMF. The apparatus may further comprise enveloping the layers with synthetic durable fabric and/or protecting fabric. The PEMF may be optional depending on daytime applications. The apparatus may further be in the form of a mattress. A method for assembling an apparatus comprising a combination of layers for protection against geopathic radiations is also provided.

### CROSS-REFERENCE TO RELATED APPLICATIONS

There are no cross-related applications.

### TECHNICAL FIELD

The present invention generally relates to the field of geopathic radiation protection devices, more particularly to mats and tiles blocking geopathic radiations and to methods for blocking geopathic radiations.

### BACKGROUND ART

Planet earth behaves like a magnet, emitting its own electromagnetic field. This field interacts with cosmic fields from the ionosphere and outer space.

Grids composed of cosmic waves and terrestrial electromagnetic waves cover the earth. Geopathic radiation includes an ultra-high frequency radiation originating in the core of the planet. It emanates out of the planet through the earth's crust and occurs in varying intensities and in varying locations on the earth's surface.

As the earth's magnetic north-south fields are basically parallel to the earth's surface, these high frequency electromagnetic waves do not appear to be directly related to the earth's magnetic field. Identification has yet to demonstrate the original source or cause of these electromagnetic waves.

The direction of emission of these electromagnetic waves is mostly perpendicular to the surface of the earth, with an angle variability of up to 45 degrees from the vertical, depending on what disturbs the electromagnetic wave path. What is created is described as “streams of distorted high-frequency radiation”.

These very high frequency electromagnetic waves are in the range of 150 GHz or greater, and their widespread occurrence typically has an adverse effect upon the human body. They are generally characterized by small wavelengths in the microwave or Nano wave frequency. The foregoing is a likely description of natural phenomena of which more research is required.

Geopathic stress consists of an invisible environment that is radiaesthetically measurable and includes water veins, fault lines, Hartmann lines, curry lines, and gamma radiation lines. Depending on the type and intensity of an intersection, a geopathic zone may be set up. Geopathic radiation zones can be assigned a value from 1 to 16 according to Gustav Von Pohl (EARTH CURRENTS—Causative Factor of Cancer & other Diseases 1932, page 29).

“Geobiology” is the investigation of the properties of electromagnetic fields of natural or artificial sources causing disturbances or interactions with organisms as well as the environment. In Geobiology terms, distortion of the natural electromagnetic field, known as “electromagnetic pollution”, can alter the electromagnetic energy of living things. In the course of evolution, living organisms have adapted to the interaction of these fields.

Geopathic stress is a problem of the environment where these fields alter human physiology. It is known that these natural electromagnetic fields have a regulatory effect on exchanges in the cell membrane.

Frequencies govern the electrical exchanges in all biological processes. Unnatural electromagnetic fields (outside a normal range) have a negative influence on cellular regulatory processes, altering them and producing a condition of stress or electrical disorder within the cells. These exposures are cumulative, and over time, subject the organism to abnormal, biological stresses.

Through scientific work, it has been confirmed by measurements in the electrical or magnetic behavior of the blood, that changes take place within these parameters when a person's place of sleep or work area is exposed to geopathic radiation which acts as a disturbance to the body.

Bio-physically and biologically, changes take place in the blood, such as sedimentation rate; body currents change over these intersections, which are known as “electromagnetic stimulation zones”, and can affect the detoxification pathways in the human body.

Telluric currents and cosmic currents systematically crisscross the planet forming a grid-like pattern with intersections that can be unfavorable to living beings. The grids exert harmful influences on humans, particularly where they interact with subterranean water veins. Any distortion of the electromagnetic lines caused by their intersection removes the correct supply of electromagnetic energy from living beings, including man.

When natural radiation is altered, it creates anomalies in ordinary radiations and magnetic fields, thus negatively impacting the human body. The stronger the intensity the less it takes of the geopathic stress to become problematic (i.e., an irritation to the system and thus adversely affect a person's health and well-being).

When a person spends a significant amount of time, e.g., sleep, or work area, geopathic radiation accumulates over time and can contribute to illnesses and conditions such as sleep disturbances, arthritis, cancer, just to name a few. It is also implicated in other illnesses by disrupting the immune system and the production of growth hormones, which repair the body during the sleep cycle.

According to research, excessive and prolonged exposure, by means of abnormal cell behavior, (resulting in abnormal cell behavior) can lead to disorders of the nervous, endocrine and immune system, which can leave the organism susceptible and can be a contributing factor among others, to cancer and other chronic degenerative illnesses.

The debilitating effects and potential contributing factor to chronic illnesses of these energy-depleting zones can, depending on other variables in a person's life, may occur immediately, or after several years, and up to two or three decades of continual exposure, depending on the intensity of the geopathic zone.

One of the health problems caused by geopathic radiation is what is known as “therapy resistance”. As long as exposure to geopathic stress is not blocked off, any form of intervention in a person's health tends to be limited.

Another negative result of geopathic radiation is that it can prevent the human body from absorbing vitamins, minerals and trace elements, and can cause allergies to foods, and the environment.

In the 1930's, Baron Gustav Von Pohl, a researcher, of Germany, determined that 95% of all cancer cases were exposed to geopathic radiation.

In the 1940's, Ernst Hartmann described global geomagnetic grids and concluded that “Cancer is a disease of location.”

In his book, “Invisible Environment”, Herbert L. Koenig states “cancer is a disease of location, and is often at the location of sleep of a cancer patient.”

Accordingly, there is a need for an object or device which may block or reduce geopathogenic radiation and electromagnetic waves from reaching humans.

### Objects of Invention

An object of the present invention is to provide an apparatus, which is very simple to create and use.

A further object of the present invention is to provide an apparatus, which is designed to help improve the quality of sleep and performance in the work place, where typically an individual spends extended lengths of time.

Another object of the present invention is to provide an apparatus, which is capable of facilitating the correct cell functioning in the human being as well as other mammals.

Another object of the present invention is to provide an apparatus for blocking and delivering electromagnetic energy, which can be manufactured with known systems and technologies.

The aim of these and other objects will become better apparent, here and after are achieved by an apparatus for blocking and delivering electromagnetic energy, characterized in that it comprises:
a) A delivery device, which in turn comprises a pulsed electromagnetic frequencies (PEMF) generator designed to deliver electromagnetic energy (waves) by means of 6 (six) coils embedded in cotton padding, with sufficient amplification to transmit through a range of thicknesses in a mattress up to 14 inches. (Fourteen)
b) A series of layers for blocking geopathic radiation from medium level up to the ultra-high frequencies (ionizing radiation).

Other and further objects and advantages of the present invention will be obvious upon an understanding of the illustrative embodiments about to be described or will be indicated in the appended claims, and various advantages not referred to herein will occur to one skilled in the art upon employment of the invention in practice.

### SUMMARY OF INVENTION

The aforesaid and other objectives of the present invention are realized by generally providing an apparatus for blocking geopathic radiation in various applications, and for returning electromagnetic energy designed to facilitate local restoration of the correct flow of electromagnetic energy during a sleep cycle of a person.

In one aspect of the invention, a protection apparatus against geopathic radiations is provided. The apparatus comprises a first layer of unleaded fabric shielding against high-frequency signals, a second layer made of material blocking medium frequency radiations, a third layer made of material for shielding against medium-to-high radiation exposure; and a protection layer, on top of any of the first, second and third layers.

The apparatus may comprise a fourth layer comprising devices for transmitting a sequence of pulsed electromagnetic frequencies (PEF) over a continual time. The devices for transmitting a sequence of PEF may comprise a plurality of coils embedded in cotton padding. The coils may be arranged in an array. The pulsed electromagnetic frequencies being produced over a continual period of 7.5 hours. The apparatus may further comprise a pulsed electromagnetic frequencies (PEMF) delivery device electrically connected to the devices for transmitting PEF.

The apparatus may further capture and drain low-level electromagnetic frequencies (EMF). The fifth layer may be made of copper mesh fabric. The fifth layer may be electrically grounded.

The apparatus may further comprise a second protection layer opposite to the first protection layer.

The first layer may be made of rubberized material. The second layer may be made of polymerized cork. The third layer may be made of metallic electromagnetic shielding fabric. The shielding fabric of the third layer may be woven. The protection layer may be made of durable or moisture resistant material.

The apparatus may be a mat, a mat under a desk or a chair of a working environment, a floor tile or may be usable with a sleeping mattress.

In another aspect of the invention, an assembly of at least two protection apparatuses is provided. In the said assembly, each of the apparatuses are connected to the PEMF delivery device.

In yet another aspect of the invention, a method for blocking geopathic radiations is provided. The method comprises blocking high-frequency signals using a first layer of unleaded fabric shielding, blocking medium frequency radiations using a second layer and blocking medium-to-high radiation exposure using a third layer.

The method may further comprise emitting a sequence of pulsed electromagnetic frequencies (PEF) over a continual time.

The emission of PEF further comprising generating the PEF using a pulsed electromagnetic frequencies generator and emitting the PEF using coils embedded in a padding. The method may further comprise capturing and draining low-level electromagnetic frequencies (EMF). The capturing of EMF may use a copper mesh. The method may further comprise sending the EMF to the ground to drain the captured EMF.

The features of the present invention which are believed to be novel are set forth with particularity in the appended claims.

### BRIEF DESCRIPTION OF DRAWINGS

The above and other objects, features and advantages of the invention will become more readily apparent from the following description, reference being made to the accompanying drawings in which:

FIG. 1 is an exploded perspective side view of an embodiment of a device for blocking electromagnetic energy in accordance with the principles of the present invention;

FIG. 2 is a top sectional orthographic projection view of the mat of FIG. 1;

FIG. 3 is an exploded perspective view of another embodiment of a mat in accordance with the principles of the present invention;

FIG. 4 is a top sectional orthographic projection view of the mat of FIG. 3;

FIG. 5 is an exploded perspective view of a floor tile in accordance with the principles of the present invention; and

FIG. 6 is a top sectional orthographic projection view of the floor tile of FIG. 5.

### DETAILED DESCRIPTION OF THE PREFERRED EMBODIMENT

A novel apparatus and method for blocking and delivering electromagnetic will be described hereinafter. Although the invention is described in terms of specific illustrative embodiment(s), it is to be understood that the embodiment(s) described herein are by way of example only and that the scope of the invention is not intended to be limited thereby.

In a preferred embodiment, shielding/blocking capability are maximized to include all degrees of intensity as well as the full range of geopathic radiation.

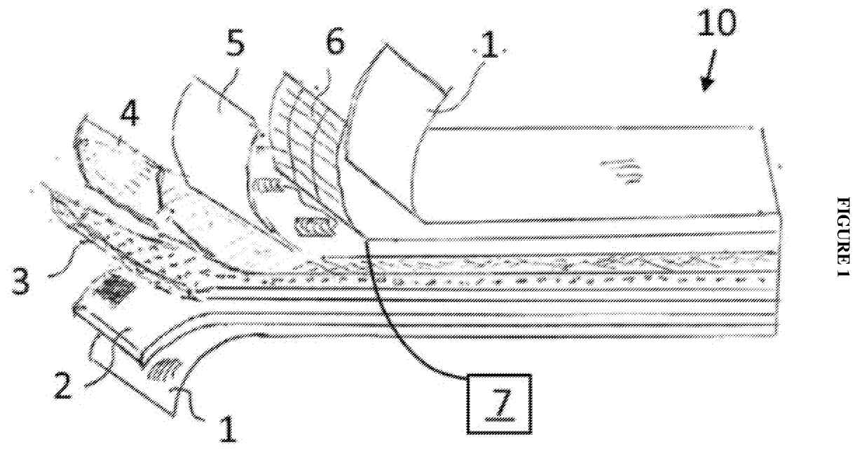

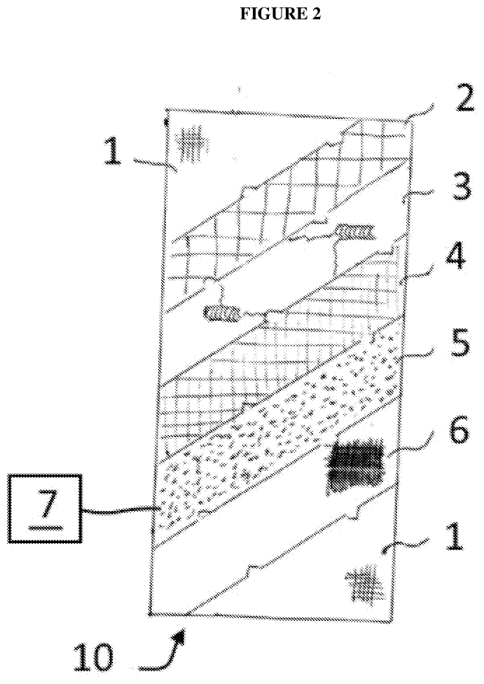

Referring to FIGS. 1 and 2, an embodiment of a multi-layered mat 10 is illustrated. The mat 10 is generally adapted for shielding from the influences of geopathogenic regions, typically in a sleeping area of a user, a working area or any area where a user may spend a substantial amount of time. The mat 10 generally comprises at least two mutually opposite outer layers 1 and at least five layers of various compositions between the two outer layers 1. The outer layers 1 are generally made of synthetic Naugahyde material. The five layers typically comprises at least one layer 2 of non-lead X Ray blocking fabric, one layer 3 of polymerized cork, one layer 4 of metallic woven electromagnetic shielding fabric, one layer of PEMF coil embedded cotton padding 5, and one layer 6 of copper net—extremely low frequency (ELF) grounding fabric.

The first, or bottom layer, 2 may be made of rubberized material used to shield against high-frequency exposure. In some embodiments, the high-frequencies range from x-ray to gamma rays. In some embodiments, the bottom layer 2 is black in color and/or has a rubber-like texture.

The second layer 3 is typically positioned directly above the bottom layer 2. The second layer 3 is made of material blocking medium frequency radiations. In some embodiments, the medium frequencies range from ultraviolet to X-rays. In a preferred embodiment, the material is a cork polymer.

The third layer 4, positioned above the second layer 3, is generally made of material used to shield against medium-to-high radiation exposure. In some embodiments, the medium-to-high frequencies range from infrared rays to visible light. In a preferred embodiment, the material of the third layer is a woven metallic fabric.

The fourth layer 5 delivers a sequence of pulsed electromagnetic frequencies over a continual period, such as but not limited to 7.5 hrs. The pulsed electromagnetic frequencies typically range between 0 to 14 Hz. In a preferred embodiment, the fourth layer 5 comprises an array of coils, the array of coils producing the pulsations of electromagnetic frequencies. The fourth layer 5 is typically connected to a PEMF 7 which control the production of pulsations.

The fifth layer 6 generally serves to capture the low-level electromagnetic frequencies (EMF) to eliminate them. In a preferred embodiment, the fifth layer 6 is made of copper mesh fabric. The EMF typically range between 50 to 60 Hz (or household electric current frequencies) at voltage between 110 to 220V. The fifth layer 6 generally eliminates the EMF through a ground, such as a connection with a ground plug (not shown). The fifth layer 6 may be in connection with the PEMF 7 to be grounded, as the PEMF 7 is grounded through a wall plug.

The above-described inner layers are preferably enveloped in durable and/or moisture resistant envelope. In some embodiment, the envelope is made of a synthetic type of durable, moisture resistant Naugahyde fabric 1. Understandably, the stacking order of each of the layers 2, 3, 4, 5 and 6 may be changed, and is within the scope of the present invention.

Rather than measuring every possible geopathically compromised location, which involves customized solutions for the consumer, the present device 10 generally aims at being applicable to most or all of the geopathic zones, regardless of type or intensity of the geopathic radiation.

The selected geopathic test zone typically comprises a confluence of most or all geopathic types of radiation, with the maximum or near maximum intensity.

The present mat 10 was tested at a chosen location, which is known as one of the most geopathically contaminated zones in Eastern Canada. As a result, the device 10 successfully blocked radiations at this geopathic zone. As such, the device 10 may potentially address all variations of types and intensities of geopathic radiation in zones of equal or lesser intensity worldwide.

A problem with a full blocking of all earth's radiations is that it leaves certain systems and cell structures deficient in electromagnetic frequencies, which are required for the maintenance of normal cell function in the human organism. Therefore, the need to reintroduce the beneficial earth energies which have been lost in the process of blocking the harmful radiations is accomplished through a PEMF delivery system. The PEMF typically comprises a proprietary combination of PEMF frequencies delivered to the body, for wellness and sleep enhancement during the sleep cycle

The mat 10 of the embodiment illustrated at FIGS. 1 and 2 may be of approximately 1 meter wide by 2 meters long, and thus suitable to be placed under, over or within a standard mattress, in the sleep area of an exposed geopathic zone. The nightly application requires the incorporation of PEMF.

Merely by way of example, the embodied panel(s) 2, 3, 4, 5 and 6 of the mat 10, which shield from the negative influences caused by alterations in magnetic fields of the earth, could be arranged inside a ready-made mattress, either above the coil springs or just beneath the pillow topper in a foam mattress.

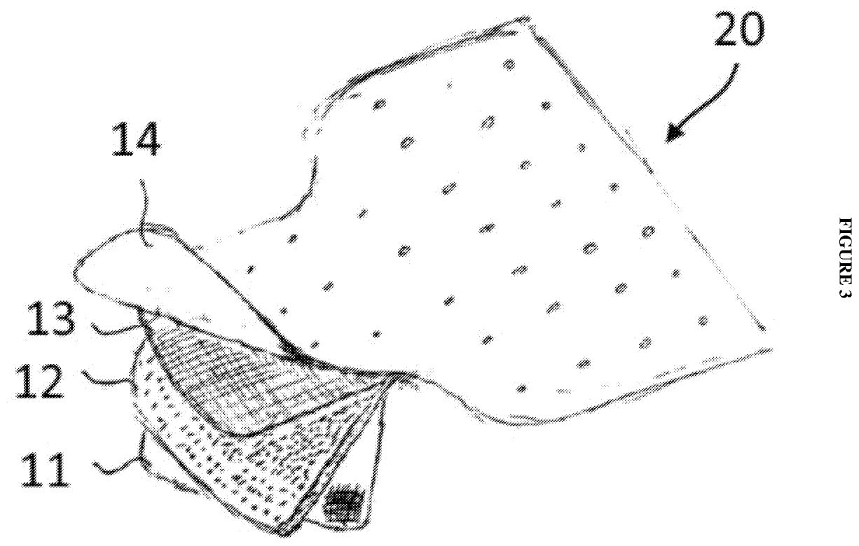

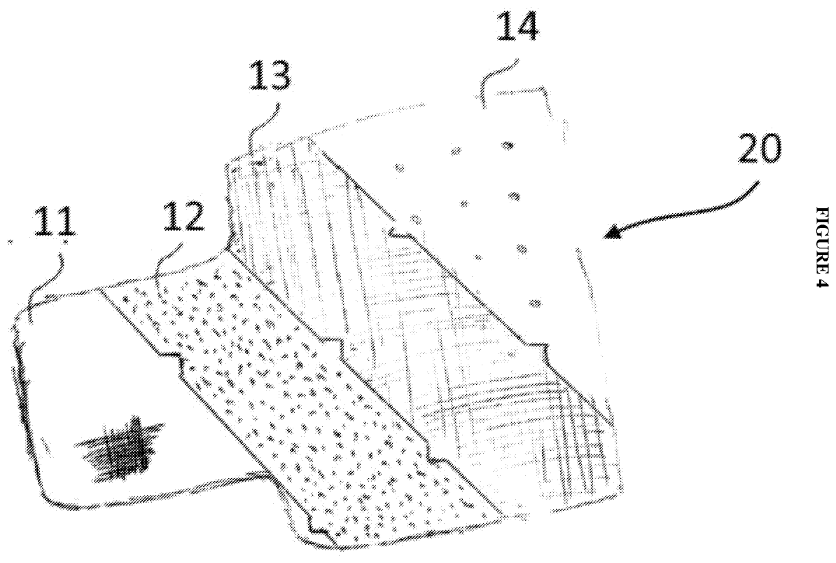

Referring now to FIGS. 3 and 4, a multilayered mat 20 for shielding from the influences of geopathogenic regions in the office area (under office desk and/or chair) is illustrated. It may be appreciated that, the device 20 used during daytime may not require PEMF.

The multi-layer mat 20 is particularly adapted for shielding from the influences of geopathogenic regions. In such an embodiment, the mat 20 comprises at least one layer 14 of rigid material, such as hard plastic. The layer 14 may be similar to carpet protector for an office chair or for any other furniture. The layer 14 is typically placed on the top of the mat 20. At least one layer 13 of metallic woven electromagnetic shielding fabric, one layer 12 of polymerized cork; and one layer 11 of non-lead or unleaded X-Ray blocking fabric may be placed underneath the mat 20.

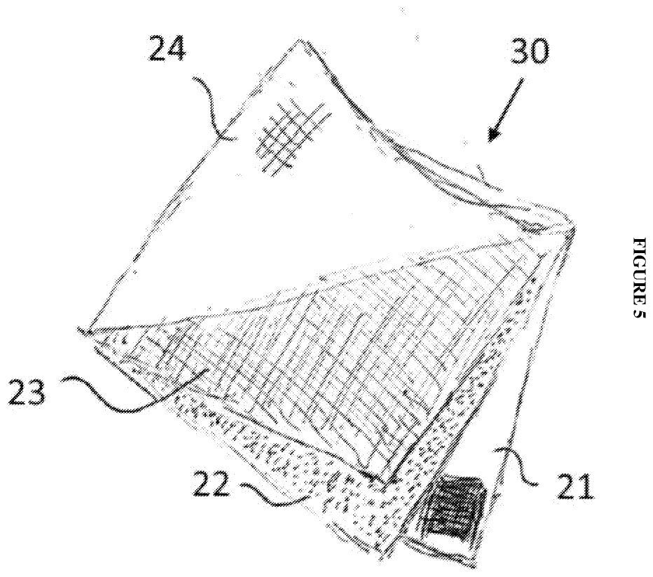

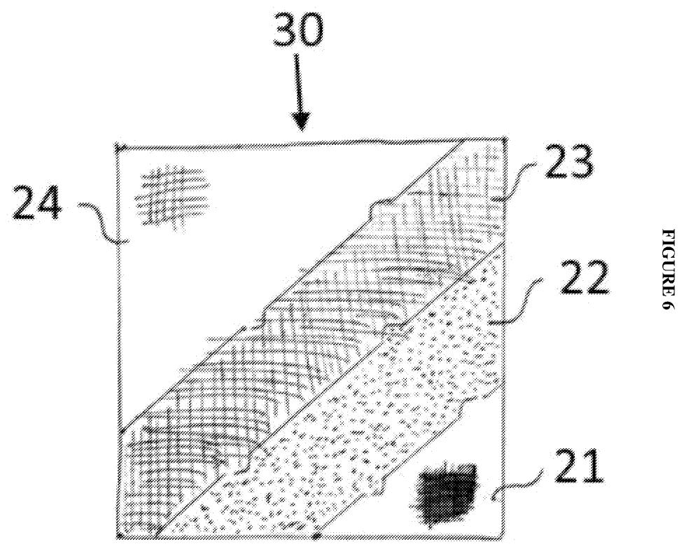

Referring now to FIGS. 5 and 6, a multilayered floor tile 30 for shielding from the influences of pathogenic regions on a floor is illustrated. In some embodiments, the floor may be in the workplace which may include, but not limited to, offices, factories, institutional buildings.

The multilayered tile 30 is particularly configured for shielding from the influences of geopathogenic regions in the workplace. The tile 30 comprises at least one layer 24 of floor tile. The tile 30 may further comprise at least one layer of metallic electromagnetic shielding fabric 23, one layer of polymerized cork 22 and one layer of non-lead X-Ray blocking fabric 21.

Understandably, the multi-layered floor mats 10, 20 or tile 30 may be used in any setting wherein a user may be affected by pathogenic and geopathogenic waves and are therefore not limited to the workplace or sleeping place only. Furthermore, a plurality of any of the apparatuses 10, 20 or 30, such as two or more, may be used together to cover a bigger area of protection. Accordingly, a single PEMF generator may be connected to more than one apparatus 10, 20 or 30.

Now referring back to FIGS. 1 and 2, an apparatus 10 for blocking and delivering electromagnetic energy is embodied as shown. The apparatus 10 comprises 2 types of devices, which operate continuously with one another, a PEMF delivery device and a plurality of 3 layers of various materials.

The PEMF delivery device 7 generally comprises a frequency generator control box, designed to power six strategically placed coils embedded above the blocking device. The plurality of three single layers of differing materials generally comprises layers, each chosen for their specific blocking of a certain range of electromagnetic radiation. It may be appreciated that the 3 layers put together are forming a broader blocking of geopathic radiation than each layer individually.

Referring now to FIGS. 3 and 4, an embodiment of an apparatus 20 for blocking geopathic radiation is shown.

The apparatus 20 comprises Geoshield Work™, a type of device which is a combination of three layers of differing materials 11, 12 and 13, each chosen for specific blocking properties for a specific range of electromagnetic radiation. The above may be bonded with contact cement beneath a standard clear, acrylic carpet protector 14 to be placed under office furniture such as an office chair. Understandably, the bonding may be performed using any other type of adhesive or cement.

Referring now to FIGS. 5 and 6, another embodiment of an apparatus 30 for blocking geopathic radiation is shown. The apparatus 30 comprises a standard floor tile type of device which is a combination of three layers of differing materials 21, 22 and 23, each chosen for their specific blocking properties for a specific range of electromagnetic radiation. The apparatus 30 may be bonded beneath a standard floor tile 24, to be placed on the floor of a work area. The bonding may be performed with bonding cement or other type of adhesive.

Conveniently, the invention provides an apparatus designed to improve the quality of life in the regions in which it is positioned since a distorted, unnatural electromagnetic field has negative influences on all existing cells and molecules.

The invention, thus conceived, is susceptible to numerous modifications and variations; all the details may further be replaced with other technically equivalent elements.

In practice, the materials employed, so long as they are compatible with the specific use, as well as the dimensions, may be any according to requirements, and to the state of the art.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1. A protection apparatus against geopathic radiations, the apparatus comprising:
a first layer of unleaded fabric shielding against high-frequency signals;
a second layer made of material blocking medium frequency radiations;
a third layer made of material for shielding against medium-to-high radiation exposure; and
a protection layer, on top of any of the first, second and third layers.

2. The apparatus of claim 1, further comprising a fourth layer comprising devices for transmitting a sequence of pulsed electromagnetic frequencies (PEF) over a continual time.

3. The apparatus of claim 2, the devices for transmitting a sequence of PEF comprising a plurality of coils embedded in cotton padding.

4. The apparatus of claim 3, the coils being arranged in an array.

5. The apparatus of claim 2, the pulsed electromagnetic frequencies being produced over a continual period of 7.5 hours.

6. The apparatus of claim 2, the apparatus further comprising a pulsed electromagnetic frequencies (PEMF) delivery device electrically connected to the devices for transmitting PEF.

7. The apparatus of claim 1 further capturing and draining low-level electromagnetic frequencies (EMF).

8. The apparatus of claim 7, the fifth layer being made of copper mesh fabric.

9. The apparatus of claim 8, the fifth layer being electrically grounded.

10. The apparatus of claim 1 further comprising a second protection layer opposite to the first protection layer.

11. The apparatus of claim 1, the first layer being made of rubberized material.

12. The apparatus of claim 1, the second layer being made of polymerized cork.

13. The apparatus of claim 1, the third layer being made of metallic electromagnetic shielding fabric.

14. The apparatus of claim 1, the shielding fabric of the third layer being woven.

15. The apparatus of claim 1, the protection layer being made of durable or moisture resistant material.

16. The apparatus of claim 1 being a mat.

17. The apparatus of claim 1 being usable with a sleeping mattress.

18. The apparatus of claim 1 being a mat under a desk or a chair of a working environment.

19. The apparatus of claim 1 being a floor tile.

20. An assembly of at least two protection apparatuses according to claim 2, wherein each of the apparatuses are connected to the PEMF delivery device.

21. A method for blocking geopathic radiations, the method:
blocking high-frequency signals using a first layer of unleaded fabric shielding;
blocking medium frequency radiations using a second layer; and
blocking medium-to-high radiation exposure using a third layer.

22. The method of claim 21 further comprising emitting a sequence of pulsed electromagnetic frequencies (PEF) over a continual time.

23. The method of claim 22, the emission of PEF further comprising generating the PEF using a pulsed electromagnetic frequencies generator and emitting the PEF using coils embedded in a padding.

24. The method of claim 21 further comprising capturing and draining low-level electromagnetic frequencies (EMF).

25. The method of claim 24, the capturing of EMF using a copper mesh.

26. The method of claim 24, the method further comprising sending the EMF to the ground to drain the captured EMF.
